(19) United States
(12) Patent Application Publication
(10) Pub. No.: US 2005/0149032 A1
(43) Pub. Date: Jul. 7, 2005

(54) Resorbable surgical fixation device

(76) Inventors: Douglas Vaughen, Downingtown, PA (US); Joseph A. Lanza, West Chester, PA (US); Ralph F. Zwirnmann, Roslyn, PA (US)

Correspondence Address: JONES DAY, 222 EAST 41ST ST, NEW YORK, NY, 10017, US

(21) Appl. No.: 10/749,345

(22) Filed: Dec. 30, 2003

(51) Int. Cl. A61B 17/58

(52) U.S. Cl. 606/77

(57) ABSTRACT

The present invention provides an improved contourable surgical fixation device that is made from a resorbable material and useful in bone reconstruction. In one embodiment, the fixation device may be made of a polymeric material. The fixation device comprises a plurality of spaced-apart fastening plates, links interconnecting the plates, and openings defined between the fastening plates by the links and fastening plates. In one embodiment, at least some of the fastening plates have fastener holes therethrough for receiving a fastener, such as a screw or tack, to secure the fixation device to the bone. The present invention provides an open-structured fixation device that is capable of being contoured in three dimensions to approximate the anatomical shape of a bone to which the fixation device may be attached.

### BACKGROUND OF THE INVENTION

The present invention generally relates to a resorbable surgical fixation device for bone reconstruction, and more particularly to a contourable mesh made from resorbable materials that is capable of being contoured in three dimensions to approximate the shape of the bone to which the mesh may be attached. The invention is particularly suited for fracture repair and reconstruction of the craniofacial skeleton, but is not limited in scope to those applications.

Biologically compatible metallic meshes capable of being formed and contoured to the three-dimensional skeletal anatomy are known for surgical use. These meshes have been employed in osteosynthesis to rejoin and repair bone discontinuities resulting from trauma (i.e., fractures) and/or surgical procedures wherein osteotomies of the bone are necessary to performing the procedure.

Various configurations of contourable meshes have been used that are commonly secured to the bone with fasteners, such as screws and tacks. One class of meshes is formed by simply punching a plurality of circular fastener openings into a generally square and flat sheet of material. These perforated sheet meshes have limited flexibility and limited three-dimensional contourability due to their generally solid or closed structures. Accordingly, they are difficult to three-dimensionally contour to irregular or intricate portions of skeletal anatomy in some cases and are prone to kinking. To eliminate the kinking problem and improve contourability, surgeons typically find it necessary to cut out multiple and/or extensive portions of such closed, solid construct perforated sheet meshes. Kinking is undesirable because it causes soft tissue irritation and other problems. FIG. 1 shows an example of such a prior art solid perforated sheet mesh having triangular relief cuts typically made by surgeons for applying this type of mesh to the frontal part of the skull. One drawback of making such customized relief cutouts is the process extends surgical time. Another drawback is that the cutouts themselves reduce the strength of the final mesh construct because the narrow section at the center of the mesh along line A-A has decreased flexural rigidity, discussed in more detail below.

In contrast to the foregoing class of solid-sheet perforated meshes, another class of more highly contourable meshes is characterized by relatively more open and flexible structures. These more flexible meshes generally have an array of circular fastener holes surrounded by generally annular rings that are interconnected by a plurality of arm-like structures joining the fastener holes together to form an open sheet-like configuration. The array of arm-like structures and fastener holes define a plurality of non-fastener openings of various configurations therebetween, thereby providing a more open mesh configuration having greater flexibility and three-dimensional contourability than the closed-structured perforated sheet meshes. These more contourable meshes are metallic, and typically are made from titanium due to the material's relatively high strength and malleability at operating room temperatures. These metallic meshes are relatively thin with representative thicknesses of only about 0.3-0.5 mm. The typical widths of the metallic arms of such meshes is equally small and may be on the order of 0.3 mm in some cases. These metallic open-structured meshes are typically secured to the bone with metallic fasteners.

The more highly contourable open-structured metallic meshes dicussed above, however, may not be suitable for all surgical applications and have some disadvantages. For example, although relatively flexible, these open-structured metallic meshes still often require some cutting or severing of the arm members during surgery to improve flexibility and allow shaping to obtain the desired final three-dimensional configuration needed to approximate the anatomical shape of the bone to which the mesh will be attached. In addition to extending surgical time, such cutting tends to leave sharp metallic burrs that can cause soft tissue irritation and patient discomfort. Still another disadvantage of metallic meshes are that the arms interconnecting the fastener holes sometimes tend to protrude upwards when contoured in three dimensions, thereby creating raised portions or points which do not lie flat against the bone, and may cause soft tissue irritation.

Significantly, a major drawback of the open-structured metallic meshes is that they sometimes require a second surgical procedure to remove the mesh after the bone has fully mended. Removal may especially be indicated in pediatric cases where the metallic mesh could interfere with normal bone growth and development if left in place. Even in adult patients, however, it is often common practice to remove metallic meshes and fasteners after the bone has mended. Allowing the metallic meshes to remain in vivo may be undesirable for other reasons, including that the meshes can sometimes be seen and felt by the patient, particularly where skin coverage over the bone is thin. Such second surgical procedures to remove the metallic mesh may be traumatic for some patients and increase the overall cost of treatment.

Implants made of biodegradable resorbable materials, particularly polymeric resorbables such as those containing lactide and/or glycolide polymers, are commonly known and used. The resorbable material will eventually dissolve over time after implantation and bone mending, thereby eliminating the need for second surgical procedures discussed above. Thus, resorbable materials have been used for implants with a generally solid structure, such as bone screws, fixation plates, and even the closed-structured perforated sheet meshes discussed above.

Polymeric resorbable implants are generally recognized as being inherently weaker in strength than their stronger metallic counterparts. Accordingly, to compensate for the disparity in strength, resorbable implants are often made larger and thicker than if the same implant were made from metal. Thus, the general perception has been that the inherently weaker resorbable polymers rendered them generally unsuitable and impractical for use in making the more intricate and delicate open-structured implants, such as the highly contourable meshes discussed above that heretofore were made of metal. Moreover, thicknesses comparable to the relatively thin open-structured metallic meshes may be difficult to achieve without sacrificing strength. Therefore, although resorbable meshes of the closed-structured perforated sheet type may have some disadvantages when applied to an irregularly and intricately contoured part of the skeletal anatomy (i.e., kinking, relief cutouts needed decreasing strength of the mesh and increasing surgical time, etc.), those type meshes continued to be widely used heretofore in such surgical applications.

Accordingly, the need exists for a resorbable mesh that could be more easily contoured to intricate and irregular shapes of the skeletal anatomy without the disadvantages of the foregoing metallic and closed-structured resorbable meshes of the past.

### SUMMARY OF THE INVENTION

The present invention is generally directed to an open-structured, highly contourable mesh made from resorbable materials. The contourable mesh comprises a plurality of spaced-apart fastening plates, deformable links interconnecting the plates, and openings interspersed between the fastening plates. The links preferably may be arcuately shaped and smoothly curved. The openings may be defined by at least a portion of the periphery of both the links and fastening plates. The openings preferably may be elongate in shape and are ordinarily not intended to receive a fastener for securing the mesh to bone. The openings provide space within the mesh construct to allow the links to be deformed in three dimensions. Accordingly, the contourable mesh of the present invention may be three-dimensionally contoured without kinking.

In one embodiment, at least some of the fastening plates may have a hole therethrough for receiving a fastener, such as a screw or tack, to secure the mesh to the bone. In another embodiment, at least some of the fastening plates do not have holes and the surgeon can add holes to the fastening plates during surgery at the desired locations. Conventional means known in the art such as drills, for example, can be used by the surgeon to add the necessary fastener holes to the fastening plates during surgery. Preferably, the fasteners used with the mesh are also made of the resorbable material; however, the fasteners may be made from a resorbable material that is different than the mesh or a non-resorbable, but preferably biocompatible material.

Unlike metallic meshes, meshes in one embodiment of the present invention advantageously are made of radiolucent resorbable materials (i.e., transparent to x-rays, radiography, CT scan, and other similar imaging techniques employed in the medical industry). Therefore, the resorbable contourable meshes are compatible with the foregoing imaging techniques, and will not interfere with such techniques when used by a medical professional to assess the status of bone healing after fracture fixation and repair.

Open-structured contourable meshes formed according to principles of the present invention may be cut from relatively thin monolithic compression molded solid sheets of resorbable polymers, with thicknesses of the sheet, for example being typically from about 0.25 mm to about 1.5 mm. Various features of the mesh (i.e., fastening plates, links, fastener openings, etc.) may be machined and/or cut into the mesh.

Resorbable open-structured meshes according to the principles of the present invention may be relatively weak in the flat two-dimensional state as cut from the solid sheet of resorbable material, but such resorbable meshes advantageously may become self-supporting by virtue of being contoured into a three-dimensional shape. The three-dimensional shaping compensates for the inherently weaker resorbable materials which gain sufficient strength for use in these type of meshes. In addition, sufficient strength is imparted to the mesh to allow open-structured, highly-contourable meshes to be made from resorbable materials with thickness comparable to similar metallic meshes. It should be noted that the three-dimensionally contoured resorbable open-structured mesh construct develops strength independent of any support provided by the bone to which it is secured.

Also importantly, it has heretofore been largely unrecognized that flexural rigidity comparable to closed-structured, solid perforated sheet resorbable meshes of the type discussed in the Background of the Invention section could be achieved with open-structured highly contourable resorbable meshes of the present invention. Flexural rigidity is one important measure of implant strength in the art. Flexural rigidity is based on the weakest part of the implants which typically coincides with that cross-sectional portion of the mesh that has the least amount of material available to resist bending moments imposed on the mesh after implantation. For the closed-structured perforated sheet prior art mesh shown in FIG. 1, for example, the weakest part occurs at the center of the mesh along line A-A which as a practical matter has been necessarily narrowed by making the triangular relief cutout modifications to three-dimensionally shape this type of sheet mesh and avoid kinking, as discussed above. At the cross-section along line A-A, there are only five sheet ligaments between the screw holes (as shown) which are available to resist bending moments imposed on the mesh. The area of coverage of this mesh is comparable to the round mesh embodiment 80 of the present invention shown in FIG. 9. By contrast, because resorbable meshes accordingly to principles of the present invention do not require relief cutouts to be three-dimensionally contoured and are not prone to kinking (discussed in detail below), a cross-section through its center which has not been narrowed by such cutouts would show that the links between the fastening plates provides much more material to resist bending moments than the five screw hole ligaments of the solid sheet perforated mesh of FIG. 1. Accordingly, meshes of the present invention can advantageously be made with a flexural rigidity at least equal to or greater than modified solid sheet-like prior art meshes, thereby obtaining the benefits of resorbable materials, but without all of the drawbacks of the solid sheet-like resorbable meshes.

In comparison to the prior art open-structured metallic meshes, advantages of the highly-contourable meshes of the present invention are also numerous. Unlike metals commonly used heretofore for the more contourable open-structured meshes described above, resorbable materials advantageously eliminate the need for second surgical procedures to remove the mesh. For instance, a resorbable mesh formed according to the principles of the present invention may be easily three-dimensionally contoured to match the anatomical shape of the bone to which it will be affixed. The resorbable meshes advantageously retain their necessary strength for a predetermined period of time following implantation (controlled by the type of resorbable material selected and other factors) to allow the bone discontinuity (resulting from a traumatic fracture and/or intentional dissection made for other surgical purposes) to mend. Then, eventually after the mesh has served its useful structural purpose of allowing the bone to fully mend, the resorbable mesh will dissolve and be absorbed by the patient's body through natural mechanisms such as hydrolysis. The resorption characteristic is especially advantageous for pediatric patients, as noted above, where bone growth is still occurring and which might otherwise be impeded by permanent metallic meshes if not removed after the bone has mended. Moreover, the resorbable mesh can be secured to the bone using resorbable fasteners, such as screws and tacks, which similarly will dissolve over time. Furthermore, the resorbable mesh can advantageously be readily cut to size without leaving sharp burrs like metal meshes. In addition, the resorbable meshes of the present invention can be shaped more easily than similar metallic meshes without having to cut the arms or links to facilitate three-dimensional shaping like often needed with the metallic meshes.

In other embodiments of a resorbable contourable mesh formed according to principles of the present invention, the mesh may comprise a plurality of spaced-apart fastening plates interconnected in a two-dimensional matrix by a plurality of arcuately-shaped deformable links that bridge the space between and connect the fastening plates to form an open-structured deformable mesh having openings interspersed therein between the links and fastening plates. The mesh is capable of being contoured in three dimensions to match the shape of a bone to which the mesh will be secured. Preferably, the openings may be elongate in shape, and in one embodiment have a narrow middle portion with a wider portion on either side. The links are preferably substantially elongate and preferably smoothly curved and arcuately shaped to avoid creating any sharp bends. Preferably, each link has a first end connectable to a first fastening plate and a second end connectable to a second fastening plate. At least some of the fastening plates preferably have a hole disposed therethrough to receive a fastener for securing the mesh to the bone. In one embodiment, at least some of the fastener holes are countersunk. In another embodiment, the mesh further comprises at least four fastening plates. The plates may be arranged in at least two rows of at least two fastening plates in each row such that the rows are arranged in spaced-apart relationship to each other.

In another embodiment, a resorbable mesh formed according to principles of the present invention may include at least two rows of spaced-apart fastening plates; each of the rows including at least two fastening plates. At least one arcuately-shaped link interconnects each of the fastening plates to at least one other fastening plate. In one embodiment, the links radiate outward from the fastening plates in a spiral pattern and the links connected to a single fastening plate are arranged in a radially spaced-apart relationship to each other. The fastening plates and links are arranged in a manner to define a plurality of elongate-shaped openings in the mesh. In another embodiment, at least some of the elongate openings are oriented vertically and at least some of the elongate openings are oriented horizontally with respect to the mesh. The fastening plates, links, and elongate openings define an open-structured mesh capable of being contoured in three dimensions to conform to the shape of a bone to which the mesh may be attached.

In one embodiment, a resorbable mesh formed according to principles of the present invention is formed from a plurality of repeating base mesh units. Each base mesh unit may comprise four spaced-apart fastening plates. In one embodiment, the fastening plates may be substantially round. The fastening plates may be equally spaced apart and arranged to form a generally square pattern. The fastening plates are preferably arranged such that each fastening plate forms a corner of the base mesh unit. In one embodiment, at least some of said fastening plates may have a hole passing therethrough to receive a fastener for attaching the mesh unit to a bone.

The base mesh unit may further comprise at least four arcuately-curved links connecting the fastening plates together. The at least four links are preferably arranged around a central opening disposed between the fastening plates such that a boundary is formed for the central opening by the links and at least a portion of the fastening plates. In one embodiment, two of the at least four arcuately-curved links project inwards toward the central opening and two of the at least four arcuately-curved links project outwards from said central opening. In another embodiment, the central opening is substantially elongate and symmetrical in shape.

The repeating base mesh unit is preferably made from a resorbable material having a glass transition temperature (T_{g}). The base mesh unit is changeable between: (a) a first condition wherein the temperature of said base mesh unit is below the glass transition temperature (T_{g}) and said base mesh unit is substantially rigid, and (b) a second condition wherein the temperature of said base mesh unit is above the glass transition temperature (T_{g}) and said base mesh unit is flexible and contourable in three dimensions to match the skeletal anatomy to which said base mesh unit may be attached.

A resorbable contourable fixation device kit is provided. The kit may comprise: (a) at least a first resorbable fixation device including a plurality of spaced-apart fastening plates, a plurality of deformable links interconnecting the fastening plates, and a plurality of elongate openings interspersed between the fastening plates, wherein the fastening plates and the links are made of a resorbable material and the fixation device is contourable in three-dimensions; and (b) a plurality of fasteners for attaching the fixation device to bone. In one embodiment, at least some of the fasteners are made from a resorbable material. Preferably, the fasteners include screws and/or tacks in another embodiment of an appropriate size to affix the fixation device to the bone. Also preferably, the first fixation device has a shape selected from the group consisting of square, round, and crescent, as described herein.

In another embodiment of a resorbable contourable fixation device kit, the kit further comprises at least a second resorbable fixation device. The second fixation device may have a different overall size (i.e., outside dimensions, as discussed herein) than the first fixation device. Alternatively, the second resorbable fixation device may have a different shape (e.g., square, round, crescent, etc.) and/or size than the first fixation device. In yet another embodiment, the kit further comprises at least a third resorbable fixation device. The kit may include without limitation a combination of any number, sizes, and/or shapes of fixation devices and fasteners for securing the devices to the bone.

Methods of contouring and attaching resorbable mesh to a bone are also provided. One method comprises the steps of: providing a resorbable mesh having a glass transition temperature (T_{g}) that is higher than average human body temperature, the mesh comprising a plurality of spaced-apart fastening plates, a plurality of arcuately-shaped deformable links interconnecting said fastening plates, the links arranged to define elongate openings between said fastening plates, and wherein the mesh is capable of being contoured in three-dimensions to conform to the shape of the bone; raising the temperature of the mesh above the glass transition temperature (T_{g}); deforming the mesh to substantially conform to the anatomical shape of the bone; cooling the temperature of the mesh to below the glass transition temperature (T_{g}); placing the mesh on the bone; and attaching the mesh to the bone. The method may further include at least some of the fastening plates have a fastener opening therethrough, providing fasteners, and inserting the fasteners through at least some of the fastener openings; wherein the fasteners are used for attaching the mesh to the bone.

In another embodiment, a method of contouring and attaching resorbable mesh to a bone comprises the steps of: providing a resorbable mesh having a glass transition temperature (T_{g}) that is higher than the average human body temperature, the mesh comprising a plurality of spaced-apart fastening plates, a plurality of arcuately-shaped deformable links interconnecting the fastening plates, the links arranged to define elongate openings between the fastening plates, and wherein the mesh is capable of being contoured in three-dimensions to conform to the shape of the bone; raising the temperature of the mesh above the glass transition temperature (T_{g}); placing the mesh on the bone; deforming the mesh to substantially conform to the anatomical shape of the bone; cooling the temperature of the mesh to below the glass transition temperature (T_{g}); and attaching the mesh to the bone. The method may further include at least some of said fastening plates have a fastener opening therethrough, providing fasteners, and inserting the fasteners through at least some of the fastener openings, wherein the fasteners are used for attaching said mesh to the bone.

It should be noted that the step of cooling the temperature of the mesh to below the glass transition temperature (T_{g}) may entail, without limitation, subjecting the heated mesh to an environment whose temperature is less than the glass transition temperature (T_{g}), such as by placing the heated mesh in a cool water or saline bath, exposing the heated mesh to ambient operating room conditions, placing the heated mesh on the bone, etc.

### BRIEF DESCRIPTION OF THE DRAWINGS

The features and advantages of the present invention will become more readily apparent from the following detailed description of the invention in which like elements are labeled similarly, and in which:

FIG. 1 is a top plan view of a solid closed-structured perforated mesh of the prior art showing typical relief cutouts made by surgeons to three-dimensionally shape the mesh to the anatomical shape of the bone;

FIG. 2 is a top plan view of a first embodiment of a contourable mesh formed according to principles of the present invention having a substantially square shape;

FIG. 3 is an enlarged view of a detail taken from FIG. 2 showing a typical repeating base mesh unit;

FIG. 4 is a cross-sectional view of one embodiment of a fastener hole taken from FIG. 2;

FIG. 5 is a top plan view of a second embodiment of a contourable mesh formed according to principles of the present invention having a substantially square shape;

FIG. 6 is an enlarged view of a detail taken from FIG. 5 showing a typical repeating base mesh unit;

FIG. 7 is a cross-sectional view of the fastener hole taken from FIG. 5;

FIG. 8 is a side view of a screw that may be used with the present invention;

FIG. 9 is a side view of a tack that may be used with the present invention;

FIG. 10 is a top plan view of a third embodiment of a contourable mesh formed according to principles of the present invention having a substantially round shape;

FIG. 11 a top plan view of a fourth embodiment of a contourable mesh formed according to principles of the present invention having a substantially crescent shape.

FIG. 12 is a perspective view of the contourable mesh of FIG. 10 formed according to principles of the present invention after it has been three-dimensionally shaped to approximate the anatomical contour of the bone; and

FIG. 13 is a perspective view of the anatomically-shaped mesh of FIG. 12 placed on a typical mounting position on a human skull.

### DESCRIPTION OF THE PREFERRED EMBODIMENTS

The description of preferred embodiments which follows is merely exemplary in nature and not intended to limit in any way the scope of the invention, its application, or uses.

The meshes of the present invention may be made from any suitable polymer. Preferably the meshes will be formed of resorbable (i.e., biodegradable and bioabsorbable) material that will eventually dissolve and be absorbed in vivo following implantation. For example, the mesh may be made from, but is not limited to, various polymers and combinations of two or more polymers to create varying copolymers, terpolymers, etc., polymer alloys and composites, polymers containing polymeric fibers of the same or different type of polymer, etc. The selection of material and individual or combinations of various polymers, methods used to manufacture the polymers and meshes, and other factors affect the functional properties of the resorbable implants, such as how long structural strength and dimensional stability is retained in vivo after implantation and the time required for complete absorption.

Meshes according to principles of the present invention may be made from resorbable materials that are crystalline or amorphous (i.e., non-crystalline) in structure depending on the specific material selected to fabricate the mesh and the method used to manufacture the mesh, both of which are a matter of design choice. The mesh manufacturing method, method of making and processing the polymer raw material (e.g., annealing, etc.), and other similar factors affect the crystallinity of both the raw material and finished product. Thus, for crystalline materials, the crystallinity of the polymer raw material and finished mesh product may be varied as a matter of design choice.

The material used to fabricate meshes according to principles of the invention may also contain or be impregnated with various additives, fillers, chemical and biologically-active agents (i.e., antibiotics, pharmaceuticals, proteins, growth factors, etc.), surface treatments, coatings, etc. to enhance the processing, manufacture, and/or performance characteristics of the materials and meshes.

Resorbable polymeric materials used in surgical implants for fracture fixation are generally somewhat rigid and inflexible at ambient operating room and human body temperatures. Inherent in such polymers, they become more readily flexible and malleable when their temperature is elevated above the glass transition temperature (T_{g}) of the material. Accordingly, resorbable meshes may be bent and contoured to match the three-dimensional shape of the bone surface to which they will be attached by heating the mesh to above the glass transition temperature (T_{g}) of the material. Means such as a hot water or saline bath, bender/cutter iron, hot air gun, or other suitable means known in the art may be used to heat the polymer. Once the resorbable mesh has been contoured and secured in place on the bone, rigidity returns as its temperature drops below the glass transition temperature (T_{g}). Preferably, the glass transition temperature (T_{g}) of the resorbable polymeric material is greater than average normal human body temperature (oral) of about 98.6 degrees Fahrenheit.

Preferably, a mesh formed according to principles of the present invention may be made from polymers such as lactides and copolymers of lactide and glycolide. More preferably, the mesh may be made of 70/30 poly (L, D/L-lactide) copolymer or 85/15 poly (L-lactide-co-glycolide) copolymer compositions.

The 70/30 poly (L, D/L-lactide) copolymer composition is a widely used resorbable polymer. Preferably, the polymer raw material of 70/30 composition has a crystallinity ranging from approximately 9.8-11.8%. The finished mesh fabricated from the 70/30 composition is preferably substantially amorphous (i.e., at least less than 10% crystalline in structure).

The copolymer raw material of the 85/15 poly (L-lactide-co-glycolide) copolymer composition (raw material) preferably has a crystallinity of approximately 18.9-32.1%. The finished mesh fabricated from the 85/15 composition is preferably substantially amorphous (i.e., at least less than 10% crystalline in structure).

Preferably, the resorbable material selected and its design configuration maintains sufficient strength in vivo for a period of time sufficient to allow the bone to mend. Preferably, meshes made from the 70/30 poly (L, D/L-lactide) copolymer composition are fully resorbed within approximately 3 years +/− after being implanted. Meshes made from the 85/15 poly (L-lactide-co-glycolide) copolymer composition are preferably fully resorbed within approximately 1 year +/− after being implanted. It will be appreciated that the thickness of the mesh and individual patient's body chemistry may affect the resorption times. It will further be appreciated that the time for the mesh to be absorbed can be varied by adjusting the composition of the mesh and its configuration.

As shown in the figures appended hereto, which are discussed in more detail below, the resorbable mesh formed according to principles of the present invention may be fabricated in a variety of shapes and sizes. Furthermore, the mesh may be cut to any shape desired in the surgical arena by preferably severing the links at various locations.

Although the resorbable mesh is preferably secured to the bone using fasteners, alternative suitable means such as adhesives may be used. If adhesives are used, the fastening plates may be provided without holes for receiving fasteners.

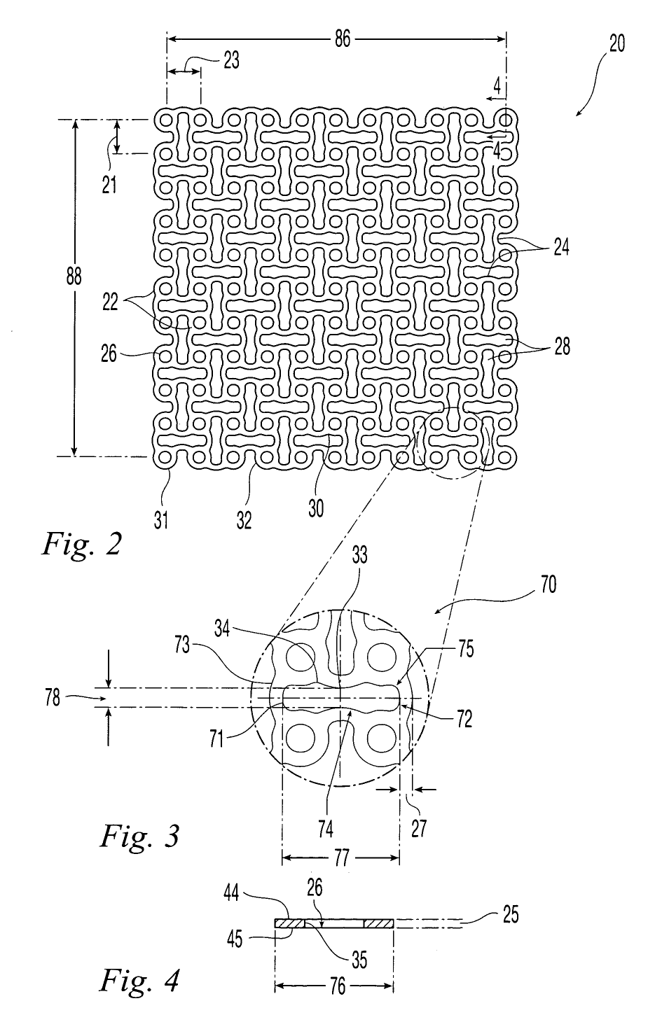

Referring now to FIG. 2, one embodiment of a resorbable mesh 20 formed according to principles of the present invention comprises a plurality of spaced-apart fastening plates 22, curved links 24 interconnecting the fastening plates 22, and openings 28 defined between the fastening plates by the links and at least a portion of the fastening plates. The fastening plates 22 are preferably generally circular or round in shape, with a preferred diameter 76 of about 3.5 mm (see FIG. 4). Other suitable diameters of fastening plates 22 may be used as a matter of design choice.

Fastening plates 22 may be separated from each other by any suitable distance and arranged in any suitable pattern, all being a matter of design choice. In general, the shorter the distances between the fastening plates 22, the stronger the mesh 20 will be because the links 24 will be concomitantly shorter and stiffer. The thickness 25 of the mesh 20 (see FIG. 4) will also influence the strength of the mesh and stiffness of the links 24, with preferred thicknesses typically being from about 0.25 mm to about 1.5 mm. Although the links and fastening plates are shown to be the same thickness, the links may be thinner or thicker than the fastening plates. It will further be appreciated that the links may preferably be relatively more flexible and deformable in comparison to the fastening plates. This helps insure that the geometry of any fastener holes formed in the fastening plates either in the factory or surgical arena remains substantially unchanged after heating and contouring the mesh.

Preferably, fastening plates 22 may be arranged and spaced in a symmetrical pattern as shown in FIG. 2 such that the horizontal distance 21 and vertical distance 23 between nearby fastening plates 22 is approximately the same. In one embodiment, typical horizontal and vertical distances 21, 23 between fastening plates 22 (center to center) may be about 5 mm. However, it should be noted that the horizontal and vertical distances 21, 23 need not be the same and also a non-symmetrical pattern may be used for fastening plates 22.

Links 24 are preferably smoothly curved and arcuately shaped to provide flexibility to mesh 20 without introducing any sharp bends which could create soft tissue irritation problems when contoured in three dimensions to approximate the anatomical shape of the bone. Accordingly, links 24 have a concave side 71 and a convex side 73. In one embodiment, links 24 may have a typical width 27 of about 0.8 mm, an inside radius of curvature 72 of about 2.0-3.0 mm, and more preferably about 2.2 mm, and an outside radius of curvature 74 of about 2.5-3.5 mm, and more preferably about 3 mm. Preferably, the transition of links 24 into fastening plates 22 is slightly rounded off with a slight radius to avoid introducing a sharp-cornered stress risers. In one embodiment, a radius of about 0.6 mm may be provided at the transition of the concave side 71 of link 24 to fastening plate 22.

As shown in FIG. 2, the number of links 24 connected to each fastening plate 22 may vary and may depend on whether the fastening plate 22 is on the exterior or interior part of mesh 20. Each fastening plate preferably has at least two links 24 connected thereto. In the embodiment shown in FIG. 2, interior fastening plates 30 preferably have at least four links 24 connected thereto, exterior corner fastening plates 31 preferably have at least two links 24 connected thereto, and exterior side fastening plates 32 preferably have at least three links 24 connected thereto. Preferably, each quadrant of fastening plate 22 has no more than one link 24. However, it should be noted that the invention is not limited in that regard and any number of links 24 may be connected to fastening plates 22 in any number of positions around the circumference of fastening plate 22. In one embodiment, the links 24 may extend or radiate outwards from the fastening plates 22 in a spiral pattern in either a clockwise and/or counterclockwise direction. Preferably, the links 24 of one fastening plate 22 all extend or radiate outwards in the same direction; however, the invention is not limited in this regard.

Openings 28, defined between fastening plates 22 by the arrangement of links 24 and fastening plates 22, may be varied in size and shape. It will be appreciated that the shape and placement of links 24 affects the shape of openings 28. Preferably, openings 28 are elongate in shape, such as that shown in FIG. 2 for example. As best seen in FIG. 3, in one embodiment, openings 28 have a narrow middle portion 33 with a wider portion 34 on either side. Such an elongate opening may have a typical length 77 of about 7.0-7.5 mm, and more preferably about 7.2 mm and a minimum width 78 (near the middle of the length) of about 1.2 mm.

Mesh 20 is preferably attached to the bone with fasteners, such as a bone screw or tack of some type. Preferably, the fasteners are made of a resorbable material which may be the same as or different than the mesh. Thus, in a preferred embodiment, at least some of the fastening plates 22 have fastener holes 26 therethrough to receive a fastener for securing mesh 20 to the bone. The size and configuration of holes 26 may be varied depending on the size and shape of the fastener to be inserted in the hole. As shown in FIG. 4, which is a cross-section taken through a fastener hole 26 of mesh 20 shown in FIG. 2, hole 26 may have straight sidewalls 35 extending between the top surface 44 and bottom surface 45 of mesh 20.

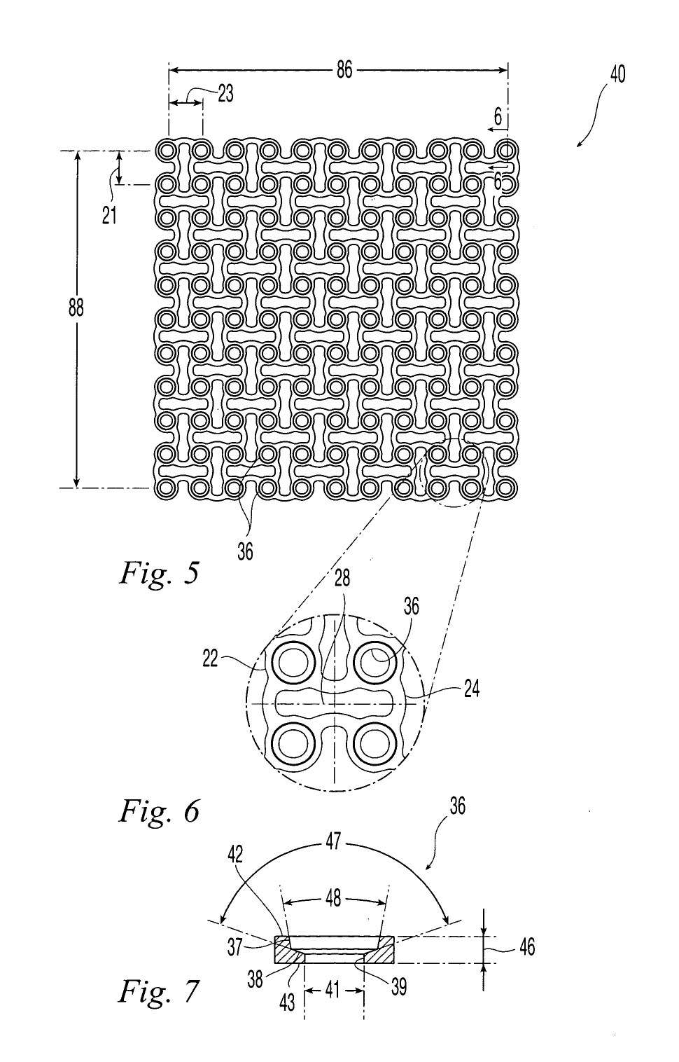

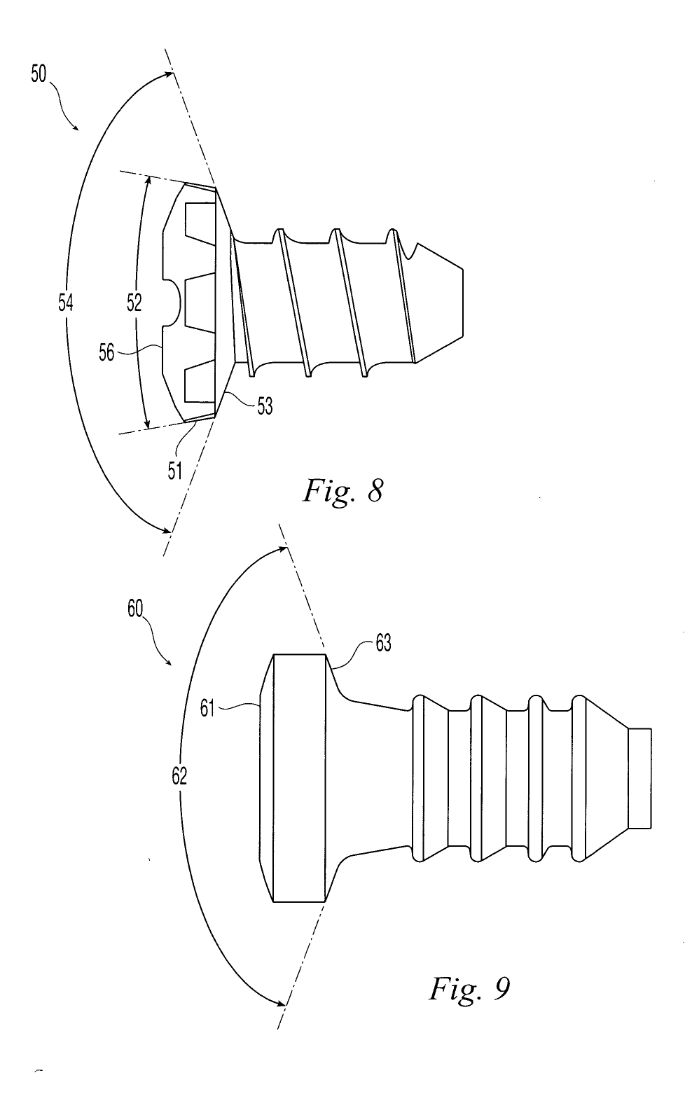

Alternatively, fastening plates 22 may be provided with a countersunk fastener hole. FIG. 7, which is a cross-section taken through a fastener hole 36 of mesh 40 shown in FIG. 5, shows one preferred embodiment of a countersunk fastener hole 36 having double-inclined walls. Starting at the top surface 42 of mesh 40, fastener hole 36 preferably comprises a first inclined wall 37, followed by an adjacent second inclined wall 38, and followed again by a straight-wall 39 which breaches the bottom surface 43 of mesh 40. First inclined wall 37 of hole 36 has a different angle 48 than second inclined wall 38 having an angle 47. Preferably, angle 48 is about 20 degrees and angle 47 is about 140 degrees in one embodiment.

It should be noted that fastener holes 22 may be of any suitable shape and are not limited to the shape described above. For example, fastener holes 22 may be conical countersunk in shape with only a single inclined wall, or hole 22 may be spherical in cross-sectional shape. Accordingly, the present invention is not limited by the shape of hole 22.

The conical countersunk holes 36 of mesh 40 shown in FIG. 7 are preferably used with fasteners having a complimentary fastener head configuration so that the head of the fastener is substantially flush with the top surface of the fastening plate 22 to reduce soft tissue irritation. For example, screw 50 shown in FIG. 8 has double-inclined surfaces on its head and may be used in holes 36. Screw 50 has a head 56 with first and second inclined surfaces 51, 53 corresponding in shape to first and second inclined walls 37, 38 of hole 36, respectively. Accordingly, inclined surfaces 51, 53 of screw head 56 have angles 52 and 54 to approximately match angles 48 and 47 of inclined walls 37, 38, respectively. In one embodiment, angles 48 and 52 are approximately 20 degrees and angles 47 and 54 are approximately 140 degrees.

Alternatively, tack 60 shown in FIG. 9 may be used in fastener hole 36 of mesh 40 (shown in FIG. 4). Tack 60 preferably has a head 61 with an inclined surface 63 that approximately matches the shape of second inclined wall 38 of fastener hole 36. Accordingly, inclined surface 63 has an angle 62 that approximately matches angle 47 of second inclined wall 38. In one embodiment, angles 47 and 62 are approximately 140 degrees.

When either screw 50 or tack 60 are inserted in hole 36 of mesh 40, the advantage this arrangement is that the head 56 or 61 respectively will be substantially flush with the top surface 42 of mesh 40 (except possibly for the slight convexity of the top of the screw or tack heads which is negligible). This helps reduce soft tissue irritation when the mesh is implanted, and the fasteners cannot be readily felt beneath the skin, particularly in locations where there is a relatively thin skin coverage over the bone.

It should be noted that screw 50 or tack 60 may also be used in straight-walled fastener hole 26 of mesh 20 (see FIG. 4). Alternatively, other fasteners (not shown) having various head configurations may also be used in fastener hole 26.

Meshes 20 (shown in FIG. 2) and 40 (shown in FIG. 5) are both preferably provided in embodiments with fastener holes 26 and 36, respectively, that are sized to accept 1.5 mm and 2.0 mm nominal size fasteners. Such meshes are commonly referred to as 1.5 mm and 2.0 mm meshes. Accordingly, in some embodiments, screws 50 and tacks 60 discussed above are also preferably provided in 1.5 and 2.0 mm nominal sizes for use in these meshes. Also preferably, a 2.5 mm nominal size screw 50 is provided to serve as an emergency screw for insertion into a 2.0 mm nominal size fastener hole in the mesh for instances when the surgeon inadvertently drills a hole that is too large in diameter for adequate purchase of bone by a 2.0 mm screw. A 2.0 mm nominal screw size 50 may serve as an emergency screw for a 1.5 mm fastener hole in the mesh.

Although it may be possible to install fasteners through openings 28 for securing mesh 20 to the bone, the fasteners are preferably installed through the fastener openings in the fastening plates which have greater strength and are less prone to failure when load is applied. It will also be appreciated that fasteners need not be installed in every fastener hole and not every fastening plate need have a fastener hole. If adhesives are used to secure the mesh to the bone, the fastening plates also need not have fastener holes. As previously discussed, the fastening plates may be provided without fastener holes and the surgeon may have such holes during surgery to the fastening plates where desired.

In some embodiments, meshes 20, 40 each preferably has a thickness 25, 46 respectively from about 0.25 mm to about 1.25. However, it should be noted that meshes 20, 40 may be of any thickness above or below the foregoing range and is a matter of design choice. It will also be appreciated that the thickness of the mesh affects parameters such as its strength and resorption time, and accordingly these factors are preferably considered when selecting the appropriate thickness for the mesh.

Referring now to FIG. 2, it will be apparent that mesh 20 is actually formed from and may be conceptualized as a plurality of individual and interconnected base mesh units each comprising an array of fastening plates 22 and links 24 which preferably form a repeatable pattern. FIG. 3 is an enlarged view taken from FIG. 2, and shows one possible embodiment of repeating base mesh unit 70 used in mesh 20 which is based on four spaced-apart fastening plates 22 connected by links 24. It should be noted that the repeating base mesh unit may have any type of fastener holes, such as straight-walled holes 26 (FIG. 4), double-inclined conical countersunk holes 36 (FIG. 7), or any other suitable shape. Alternatively, fastening plates 22 may not have any holes for example if the mesh is to be secured to the bone using adhesives, if holes are to be added during surgery, or if fasteners are inserted through openings 28 to attach the mesh to the bone.

It will be appreciated that repeating base mesh unit 70 may have any number of links 24 connecting the fastening plates 22 together as a matter of design choice, and may depend in part on whether the mesh unit will be on the exterior or interior of the mesh, as discussed above. Accordingly, the number of links 24 associated with each fastening plate in repeating base mesh unit 70 can be varied and does not limit the invention in any way.

By manufacturing and interconnecting various numbers of repeating base mesh units 70 in various arrangements or layouts, a multitude of mesh shapes and sizes are possible. For example, meshes 20 and 40 shown in FIGS. 2 and 5, respectively, are generally square in shape and for convenience may be referred to as square meshes. These square meshes in some embodiments may typically measure in outside dimensions from about 20 mm×20 mm square to about 150 mm×150 mm square (i.e., outside width 86×outside length 88 measured from fastener hole to fastener hole).

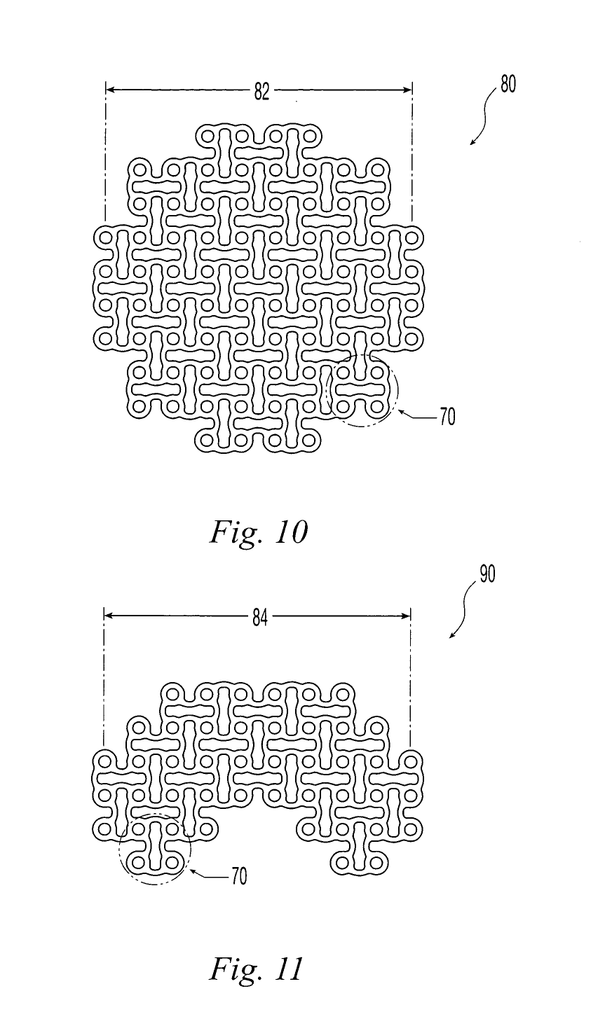

FIG. 10 illustrates a mesh 80 with a generally circular configuration formed from repeating base mesh units 70. Mesh 80 may conveniently be referred to as a round or diameter mesh. These round meshes in some embodiments may typically measure from about 20 mm in diameter 82 to about 150 mm in diameter 82 (outside dimensions). FIG. 11 illustrates a crescent-shaped mesh 90 comprised of repeating base mesh unit 70. These crescent-shaped meshes in some embodiments may typically measure in length 84 from about 45 mm to about 75 mm (outside dimensions).

Although FIGS. 10 and 11 show straight-walled fastener holes 26, it will be appreciated that the fastener holes may alternatively be double-inclined conical countersunk holes like holes 36 shown in FIG. 7 or any other suitable shape.

It should be noted that numerous shapes, sizes, and/or thicknesses of meshes may be constructed by varying the number and manner in which repeating base mesh units are interconnected. Accordingly, the invention is not limited to the shapes and sizes described herein which are presented only as an illustration of some of the configurations possible.

Another method which may be used to create various shapes and sizes of meshes can be accomplished by the surgeon in the surgical arena by removing various fastening plates 22 and links 24 to create mesh shapes particularly suited to the specific needs of an individual patient. Accordingly, the surgeon may begin with any convenient two-dimensional mesh shape and then modify that shape to suit in the foregoing manner using surgical scissors or snips.

A resorbable contourable fixation device kit is provided and will now be described. The kit may comprise: (a) at least a first resorbable fixation device including a plurality of spaced-apart fastening plates, a plurality of deformable links interconnecting the fastening plates, and a plurality of elongate openings interspersed between the fastening plates, wherein the fastening plates and the links are made of a resorbable material and the fixation device is contourable in three-dimensions; and (b) a plurality of fasteners for attaching the fixation device to bone. In one embodiment, at least some of the fasteners are made from a resorbable material. Preferably, the fasteners include screws and/or tacks in another embodiment of an appropriate size to affix the fixation device to the bone. Also preferably, the first fixation device has a shape selected from the group consisting of square, round, and crescent, as described above.

In another embodiment of a resorbable contourable fixation device kit, the kit further comprises at least a second resorbable fixation device. The second fixation device may have a different overall size (i.e., outside dimensions, as discussed above) than the first fixation device. For example, without limitation, the kit may include a plurality of square meshes preferably ranging in size from 20 mm×20 mm to 150 mm×150 mm or larger. Alternatively, the second resorbable fixation device may have a different shape (e.g., square, round, crescent, etc.), overall size, and/or thickness than the first fixation device. In yet another embodiment, the kit further comprises at least a third resorbable fixation device. The third fixation device also may have a different shape, overall size, and/or thickness than the first or second fixation devices. Accordingly, it will be appreciated that the kit may include without limitation a combination of any number, sizes, and/or shapes of fixation devices and fasteners for securing the devices to the bone.

A method of contouring and implanting a resorbable contourable mesh formed according to principles of the present invention will now be described with reference to FIG. 2 and mesh 20 for convenience. Mesh 20, preferably housed in sterile packaging and having the features described above, is provided to the surgeon in its initial rigid and flat two-dimensional form. In the surgical arena, the surgeon preferably first determines the implant reception site on the bone and the final three-dimensional shape of mesh 20 based on the anatomical three-dimensional shape of reception site. The surgeon next heats resorbable mesh 20 to above its glass transition temperature (T_{g}) to make the mesh malleable by any suitable means commonly known in the art, such as a hot water or saline bath, hot air gun, bender/cutter iron, etc., as discussed above. Preferably, the glass transition temperature (T_{g}) is above ambient operating room and human body temperatures. In one embodiment, the resorbable material from which mesh 20 is made may have a glass transition temperature (T_{g}) of about 50-55 degrees C. or above. Mesh 20 may then be placed directly on the bone reception site and contoured to the desired three-dimensional shape by the surgeon. Mesh 20 is now cooled by allowing its temperature to fall below the glass transition temperature (T_{g}) whereupon the mesh returns to its rigid condition and holds the three-dimensional contoured shape. Alternatively, mesh 20 may first be contoured to the desired three-dimensional shape prior to being placed on the bone, and then cooled to below the glass transition temperature (T_{g}) whereupon the mesh returns to its rigid condition and holds the three-dimensional contoured shape. The cooling step not only produces the required final shape of mesh 20, but also results in a three-dimensional mesh construct having greater strength than its initial flat two-dimensional form. Thus, the final three-dimensional mesh 20 construct has sufficient strength to resist in vivo loads without failure.

It should be noted that if the surgeon elects the alternative step noted above of shaping the heated mesh before placing or applying it to the bone, the process of heating and shaping the mesh may be repeated until the surgeon is satisfied that the three-dimensional shape of the mesh adequately matches the anatomical shape of the bone. Preferably, the reheating process is limited to up to about ten times. Also preferably, the duration of the heating step (i.e., time held above the glass transition temperature (T_{g})) to sufficiently heat the mesh for shaping is about ten seconds.

Once the surgeon is satisfied with the three-dimensional shape of mesh 20, a sufficient number of holes are next drilled into the bone at various locations to preferably receive resorbable fasteners, such as without limitation bone screws 50 or tacks 60 described herein. Preferably, the holes may be drilled with mesh 20 in place on the bone to facilitate properly locating the holes into the bone. If drilled without mesh 20 on the bone, mesh 20 is then placed and positioned onto the bone to line up the fastener holes 26 with the pre-drilled bone-receiving holes. In either case, fasteners are then inserted through fastener holes 26 and into the pre-drilled bone-receiving holes to secure mesh 20 to the bone.

Alternatively, if mesh 20 is supplied with fastening plates 22 that do not have prefabricated fastener holes made at the factory as discussed above, the surgeon may add fastener holes to the fastening plates 22 where desired by means such as drilling, for example. The process of then drilling bone-receiving holes into the bone and inserting fasteners through the fastener holes into the bone may be carried out in the manner described above. Holes may also be drilled through fastening plates 22 and the bone at the same time while mesh 20 is placed on the bone.

If adhesives are used to attach mesh 20 to the bone, the above steps involving drilling holes into the bone and inserting fasteners through the mesh into the bone-receiving holes may be skipped. Instead, after the final desired three-dimensional shape of mesh 20 has been produced, adhesive is applied to at least some of fastening plates 22 (which alternatively need not have fastener holes 26 and/or 36 in this case). Mesh 20 is then placed on the bone and the adhesive-laden fastening plates are put into contact with the bone.

Alternatively, another possibility of attaching mesh 20 to the bone using adhesives includes providing mesh 20 with predrilled holes 26 and/or 36, or drilling holes through fastening plates 22 in the surgical arena. Adhesive is then inserted through the holes with mesh 20 in place on the bone. Preferably, the adhesive may be of the type that will adhere to the bone and harden to a solid upon curing, thereby forming a rivet-like attachment through the fastening plate 22 holes to secure mesh 20 to the bone. This creates fixation between mesh 20 and the bone by both adhesive and mechanical means. In one embodiment, fastener holes like or similar to holes 36 (see FIG. 7) with a top opening that is wider than the bottom bone-contacting opening may preferably be used which will form rivet-like heads once the hardening adhesive cures.

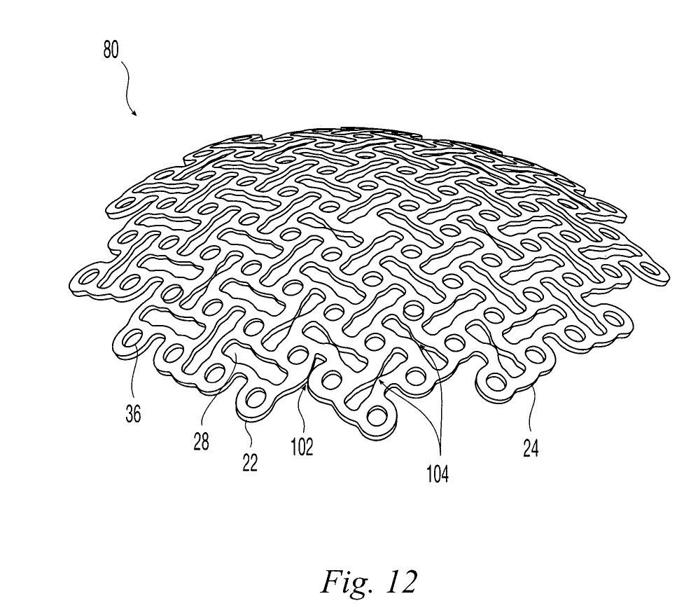

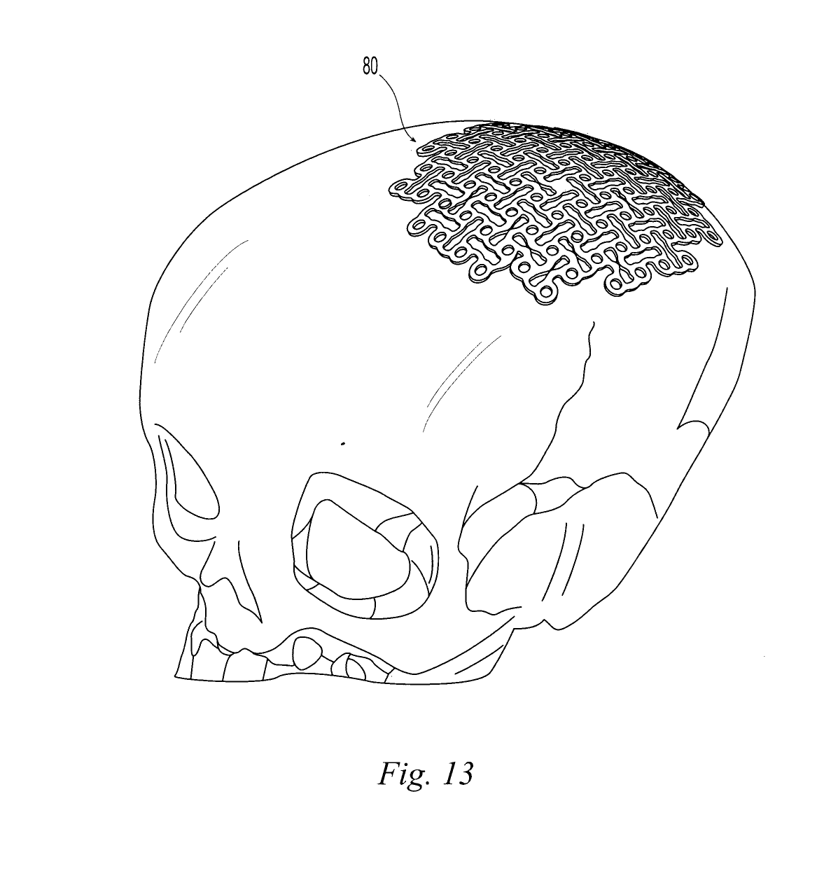

FIG. 12 shows resorbable mesh 80 formed according to principles of the present invention that is in its final three-dimensionally contoured and rigid shape (i.e., temperature of the mesh below the glass transition temperature (T_{g}) of the resorbable material used). Mesh 80 may be applied to a top area of the skull, as shown in FIG. 13, or any other portion of the skull as appopriate. As discussed above, the final three-dimensional shape of mesh 80 develops suitable strength to resist in vivo loads applied to the mesh 80 following implantation. Besides it three-dimensional shape, mechanical interference between mutually contacting structural features of mesh 80 further contributes to and adds strength to the mesh. For example, as shown in FIG. 12, some of the normally spaced-apart fastening plates 22 and links 24 may come into contact with each other as shown at location 102 depending on the three-dimensional contour of mesh 80. In addition, some links 24 may also come into contact with each other as shown at locations 104. Accordingly, such contact between the fastener holes 22 and links 24 beneficially increases the rigidity and concomitantly the strength of the mesh 80 structure. In addition, the ability of mesh 20 to deform in the manner just described also allows a final mesh shape having a greater and/or steeper depth to be created which is advantageous in certain anatomical skeletal areas requiring the mesh to be intricately shaped, such as the region of the skull near the bridge of the nose for example.

It should be noted that although mesh 80 shows countersunk fastener holes 36 of the type shown in FIG. 7, plain holes or any other suitable type of fastener hole may be used.

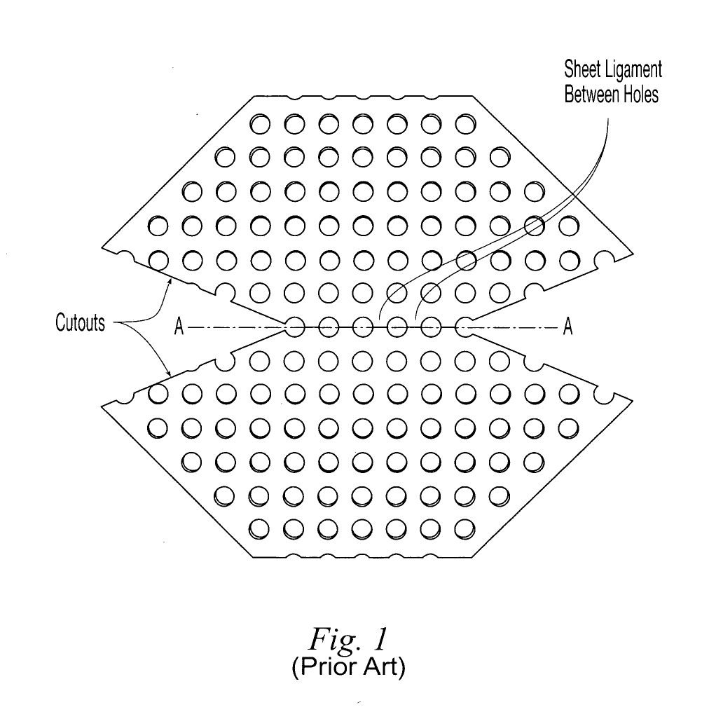

One of the many advantages of the resorbable contourable mesh accordingly to principles of the present invention is that it can conform to the shape of the skeletal anatomy without producing wrinkles or kinks during contouring like closed-structured perforated sheet type meshes of the prior art, such as that shown in FIG. 1. As discussed above, these prior art meshes require surgeons to make relief cutouts (see FIG. 1) in the mesh to avoid undesirable kinks, which may especially become extreme in anatomical skeletal areas of complex geometry. Creating such cutouts are cumbersome for surgeons and increases surgical time, both problems of which are overcome by the highly contourable resorbable mesh of the present invention which resists kinking when contoured. One mechanism that eliminates the need to cut meshes of the present invention is the ability of links 24 to move and adjust as required during the three-dimensional contouring step. For example, as shown for mesh 80 in FIG. 12 after contouring, some of the initially spaced-apart links 24 may move into contact with each other (see location 104) or into contact with some of the fastener holes (see location 102). Accordingly, in lieu of kinking like the prior art closed-structured meshes having no non-fastener openings between fastener holes, the links 24 of mesh 100 may be displaced into elongate openings 28, thereby avoiding a kinking problem. It is thus apparent that elongate openings 28 provide space or zones within the mesh 80 construct to accept displacement of the links 24 or fastening plates 22 within the plane of mesh 80. Therefore, it should be noted that the shape of some of the openings 28 whose shape was symmetrical before contouring are no longer symmetrical in shape after contouring due to the movement of links 24 and/or fastening plates 22 into openings 28.

Preferably, the resorbable contourable mesh of the present invention is produced from a compression-molded flat solid sheet of resorbable polymer, preferably the 70/30 lactide or 85/15 lactide-glycolide copolymer compositions described above. The mesh and its various structural features (i.e., fastener holes 26 and 36, links 24, elongate openings 28, etc.) preferably may be made by machining and/or cutting the flat polymeric sheet using any suitable means, including end mills, reamers, cutters, drills, and/or similar cutting tooling.

It should be noted that additional suitable means of manufacturing the resorbable contourable mesh according to principles of the present invention are contemplated and may be used alone or in combination, such means being known to those skilled in the art. For example, without limitation, the resorbable contourable mesh may be produced by punching or stamping (using single or progressive die stamping processes known in the art), high pressure water cutting, laser cutting, etc. Accordingly, the invention is not limited in any way by the means used to manufacture the resorbable contourable mesh.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

1. A resorbable contourable fixation device comprising:
a plurality of spaced-apart fastening plates; and
a plurality of deformable links interconnecting said fastening plates;
wherein said fastening plates and said links are made of a resorbable material, said fixation device being contourable in three-dimensions.

2. The resorbable fixation device of claim 1, further comprising a plurality elongate openings interspersed between said fastening plates.

3. The resorbable fixation device of claim 1, wherein said links have a smoothly curved arcuate shape.

4. The resorbable fixation device of claim 3, wherein said arcuate links have a width of about 0.8 mm, an inside radius of curvature of about 2.2 mm and an outside radius of curvature of about 3 mm.

5. The resorbable fixation device of claim 1, wherein said fastening plates do not deform when said fixation device is contoured.

6. The resorbable fixation device of claim 1, wherein said fixation device is formed from a monolithic single sheet of material.

7. The resorbable fixation device of claim 6, wherein said sheet of material is compression molded.

8. The resorbable fixation device of claim 1, further comprising at least some of said fastening plates having holes therethrough to receive a fastener for securing said fixation device to the bone.

9. The resorbable fixation device of claim 8, wherein at least one of said fastener holes is a countersunk hole.

10. The resorbable fixation device of claim 9, wherein said countersunk hole further comprises double inclined walls including a first inclined wall forming a first wall angle and a second inclined wall forming a second wall angle, said first and second angles being different.

11. The resorbable fixation device of claim 10, wherein said first angle is about 20 degrees and said second angle is about 140 degrees.

12. The resorbable fixation device of claim 10, further comprising a fastener made of a resorbable material, said fastener having a head including a first inclined surface forming a first head angle and a second inclined surface forming a second head angle,
wherein said first and second head angles are configured and arranged to substantially match said first and second wall angles of said countersunk fastener hole.

13. The resorbable fixation device of claim 1, wherein said fastener holes are spaced at a distance of about 5 mm from each other measured from center to center of said fastener holes.

14. The resorbable fixation device of claim 1, wherein said fastening plates are generally round in shape.

15. The resorbable fixation device of claim 14, wherein a portion of the periphery of four of said fastening plates and a portion of the periphery of four links forms elongate openings in said fixation device.

16. The resorbable fixation device of claim 15, wherein said elongate openings have a length of about 7.0 mm to about 8.0 mm and a minimum width of about 1.0 mm to about 1.5 mm.

17. The resorbable fixation device of claim 15, wherein said elongate openings before contouring are symmetrical in shape.

18. The resorbable fixation device of claim 17, wherein at least some of said elongate openings after contouring in at least two planes are not symmetrical in shape.

19. The resorbable fixation device of claim 18, wherein at least some of said links contact each other or the fastening plates after contouring, thereby increasing the rigidity of said fixation device.

20. The resorbable fixation device of claim 1, further comprising each of said links having a first end connectable to one of said fastening plates and a second end connectable to a different said fastening plate.

21. The resorbable fixation device of claim 20, wherein said links are connected to said fastening plates such that said links extend radially outwards from said fastening plates in a spiral pattern.

22. The resorbable fixation device of claim 21, further comprising said links having a concave side, wherein said connection between said concave side of said links and said fastening plates has inside radius of about 0.6 mm.

23. The resorbable fixation device of claim 1, wherein said fixation device has a thickness of about 0.25 mm to about 1.2 mm.

24. The resorbable fixation device of claim 1, wherein said fixation device has a substantially square shape.

25. The resorbable fixation device of claim 24, wherein said fixation device is about 20 mm×20 mm square to about 150 mm×150 mm square.

26. The resorbable fixation device of claim 1, wherein said fixation device has a substantially round shape.

27. The resorbable fixation device of claim 26, wherein said fixation device has a diameter from about 20 mm to about 150 mm.

28. The resorbable fixation device of claim 1, wherein said fixation device has a substantially crescent shape.

29. The resorbable fixation device of claim 28, wherein said fixation device has a length of about 45 mm to about 75 mm.

30. The resorbable fixation device of claim 1, further comprising at least two rows of spaced-apart fastening plates, each of said rows including at least two fastening plates.

31. The resorbable fixation device of claim 1, wherein said resorbable material contains lactide.

32. The resorbable fixation device of claim 31, wherein said resorbable fixation device further comprises glycolide.

33. A resorbable fixation device capable of being secured to bone, said fixation device comprising:
a plurality of spaced-apart fastening plates, at least some of said fastening plates having a fastener hole therethrough to receive a fastener for securing said fixation device to the bone;
a plurality of arcuately-shaped links interconnecting said fastening plates and extending from said fastening plates in a spiral pattern;
wherein said fastening plates and links are made of a resorbable material,
wherein a plurality of said fastening plates are interconnected by four links, said interconnected fastening plates and links form an open-structured deformable fixation device having elongate openings therein, said fixation device capable of being contoured in three dimensions.

34. The resorbable fixation device of claim 33, wherein at least some of said fastener holes are countersunk.

35. The resorbable fixation device of claim 33, wherein said fixation device further comprises at least four fastening plates, said plates arranged in at least two rows of at least two fastening plates in each row, said rows arranged in spaced-apart relationship to each other.

36. A resorbable contourable fixation device comprising:
at least two rows of spaced-apart fastening plates, each of said rows including at least two fastening plates;
at least one arcuately-shaped link interconnecting each of said fastening plates to at least one other fastening plate;
said fastening plates and links arranged to define a plurality of elongate-shaped openings in said fixation device;
wherein said fastening plates and said links are formed of a resorbable material, and
whereby said fastening plates, links, and elongate openings define an open-structured fixation device capable of being contoured in three dimensions.

37. The resorbable fixation device of claim 36, wherein at least some of the elongate openings are oriented vertically and at least some of said elongate openings are oriented horizontally with respect to said fixation device.

38. The resorbable fixation device of claim 36, wherein said links radiate outward from said fastening plates in a spiral pattern.

39. The resorbable fixation device of claim 36, wherein said resorbable material is a copolymer containing lactide.

40. The resorbable fixation device of claim 36, wherein said resorbable material is a copolymer of lactide and glycolide.

41. A resorbable contourable fixation device formed from a plurality of repeating base fixation device units, each said base fixation device unit comprising:
four spaced-apart fastening plates, said fastening plates arranged such that each said fastening plate forms a corner of said base fixation device unit, at least some of said fastening plates having a hole passing therethrough to receive a fastener for attaching said base fixation device unit to a bone;
at least four arcuately-curved links connecting said fastening plates together, said at least four links arranged around an opening formed by said at least four links and at least a portion of said fastening plates;
wherein said base fixation device is made from a resorbable material having a glass transition temperature (T_{g});
whereby said base fixation device unit is changeable between:
a) a first condition wherein the temperature of said base fixation device unit is below the glass transition temperature (T_{g}) and said base fixation device unit is substantially rigid, and
b) a second condition wherein the temperature of said base fixation device unit is above the glass transition temperature (T_{g}) and said base fixation device unit is flexible and contourable in three dimensions.

42. The resorbable fixation device of claim 41, further comprising said fastening plates being substantially round in shape.

43. The resorbable fixation device of claim 41, wherein said fastening plates are equally spaced apart so as to form a substantially square shape.

44. The resorbable fixation device of claim 41, wherein two of said at least four arcuately-curved links project inwards toward said opening and two of said at least four arcuately-curved links project outwards from said opening.

45. The resorbable fixation device of claim 44, wherein said opening is substantially elongate and symmetrical in shape.

46. A method of contouring and attaching a resorbable fixation device to a bone comprising the steps of:
providing a resorbable fixation device having a glass transition temperature (T_{g}) that is higher than average human body temperature, said fixation device comprising:
a) a plurality of spaced-apart fastening plates;
b) a plurality of arcuately-shaped deformable links interconnecting said fastening plates, said links arranged to define elongate openings between said fastening plates, said fixation device capable of being contoured in three-dimensions to conform to the shape of the bone;
raising the temperature of said fixation device above the glass transition temperature (T_{g});
deforming said fixation device to substantially conform to the anatomical shape of the bone;
cooling the temperature of the fixation device to below the glass transition temperature (T_{g});
placing said fixation device on the bone; and
attaching said fixation device to the bone.

47. The method of claim 46, wherein at least some of said fastening plates have a fastener hole therethrough, and further comprising the steps of:
providing fasteners;
inserting said fasteners through at least some of said fastener holes,
wherein said fasteners are used for attaching said fixation device to the bone.

48. The method of claim 46, further comprising the steps of:
forming fastener holes in at least some of said fastening plates;
providing fasteners;
inserting said fasteners through at least some of said fastener holes,
wherein said fasteners are used for attaching said fixation device to the bone.

49. A method of contouring and attaching a resorbable fixation device to a bone comprising the steps of:
providing a resorbable fixation device having a glass transition temperature (T_{g}) that is higher than the average human body temperature, said fixation device comprising:
a) a plurality of spaced-apart fastening plates;
b) a plurality of arcuately-shaped deformable links interconnecting said fastening plates, said links arranged to define elongate openings between said fastening plates, said fixation device capable of being contoured in three-dimensions to conform to the shape of the bone;
raising the temperature of said fixation device above the glass transition temperature (T_{g});
placing said fixation device on the bone;
deforming said fixation device to substantially conform to the anatomical shape of the bone;
cooling the temperature of the fixation device to below the glass transition temperature (T_{g}); and
attaching said fixation device to the bone.

50. The method of claim 49, wherein at least some of said fastening plates have a fastener hole therethrough, and further comprising the steps of:
providing fasteners;
inserting said fasteners through at least some of said fastener holes,
wherein said fasteners are used for attaching said fixation device to the bone.

51. The method of claim 49, further comprising the steps of:
forming fastener holes in at least some of said fastening plates;
providing fasteners;
inserting said fasteners through at least some of said fastener holes,
wherein said fasteners are used for attaching said fixation device to the bone.

52. A resorbable contourable fixation device kit comprising:
at least a first resorbable fixation device comprising:
a) a plurality of spaced-apart fastening plates;
b) a plurality of deformable links interconnecting said fastening plates; and
c) a plurality elongate openings interspersed between said fastening plates, wherein said fastening plates and said links are made of a resorbable material, said fixation device being contourable in three-dimensions; and
a plurality of fasteners for attaching said fixation device to bone.

53. The kit of claim 52, wherein said links have a curved arcuate shape.

54. The kit of claim 53, wherein said links extend radially outward from said fastening plates in a spiral pattern.

55. The kit of claim 52, wherein at least some of said fastening plates have a fastener hole therethrough.

56. The kit of claim 52, wherein at least some of said fasteners are made from a resorbable material.

57. The kit of claim 52, wherein said fasteners include screws or tacks.

58. The kit of claim 52, wherein said at least first fixation device has a shape selected from the group consisting of square, round, and crescent.

59. The kit of claim 52, further comprising at least a second resorbable fixation device, said second fixation device having a different overall size than said at least first fixation device.

60. The kit of claim 52, further comprising at least a second resorbable fixation device, said second fixation device having a different shape than said at least first fixation device.

61. The kit of claim 52, further comprising at least a second resorbable fixation device, said second fixation device having a different thickness than said at least first fixation device.

62. The kit of claim 60, further comprising at least a third resorbable fixation device, said third fixation device having a different shape than said at least first and second fixation devices.

63. The resorbable fixation device of claim 1, further comprising the resorbable material being radiolucent.
